(19) United States
(12) Patent Application Publication
(10) Pub. No.: US 2014/0277358 A1
(43) Pub. Date: Sep. 18, 2014

(54) STRIPED STENT INTRODUCER

(71) Applicant: DEPUY SYNTHES PRODUCTS, LLC, Raynham, MA (US)

(72) Inventor: ROBERT SLAZAS, Pinecrest, FL (US)

(73) Assignee: DePuy Synthes Products, LLC, Raynham, MA (US)

(21) Appl. No.: 13/801,283

(22) Filed: Mar. 13, 2013

(51) Int. Cl. A61F 2/95

(52) U.S. Cl. CPC A61F 2/95
USPC 623/1.12

(57) ABSTRACT

A tubular introducer sheath is made of predominantly clear material, with a colored stripe that extends along the length of the tubular introducer sheath, and that preferably spirals along the length of the tubular introducer sheath around the longitudinal axis of the tubular introducer sheath. The colored stripe has a width that is sufficiently wide to be visible externally, and is sufficiently narrow that the clear area allows visual inspection through the tubular introducer sheath. The distal end portion of the tubular introducer sheath may also be tapered.

### BACKGROUND OF THE INVENTION

This invention relates generally to devices for interventional therapeutic treatment or vascular surgery for treatment of defects in the vasculature, and more particularly concerns a system and method for delivering endovascular medical devices, such as for treatment of aneurysms.

When introducing an endovascular medical device into the proximal hub of a catheter (through which the device is intended to travel), there is an important step that can be difficult for the user to complete successfully. It is important that the user be able to visually inspect the device while it is enclosed in a tubular introducer sheath, to ensure that it is not deranged or damaged. It is also important that the user inserts the introducer sheath all the way to the bottom of the tapered opening of the proximal hub of the catheter, so that the device is pushed directly into the lumen of the catheter. However, when using introducer sheaths made of entirely clear material it is difficult to tell if they have been inserted all the way to the bottom of the tapered opening of the hub. Since endovascular procedures are typically done with “saline flush” (the catheter and hub are filled with water), introducers made of clear material tend to disappear when submerged and the edges are very hard to distinguish.

One attempt to solve this problem has involved adding a colorant to the entire material of the introducer sheath, making a sort of “tinted” appearance. This improves the ability to detect the end of the introducer sheath, but detracts from the user's ability to inspect the device through the introducer sheath. In fact, the two requirements are in direct competition. The easier to inspect, the harder to detect the introducer sheath, and vice versa.

Another attempt to solve this problem involved adding a colored tip to the introducer sheath. This leaves a large portion of the introducer sheath clear for inspection, while coloring a distal section for detection. This approach takes the two requirements out of competition with each other, but adds a new risk of tip separation and additional cost of manufacturing the discrete tip onto the introducer sheath body.

It would be desirable to provide a tubular introducer sheath that allows a user to determine if the distal end of the introducer sheath has been inserted all the way to the bottom of a tapered hub opening, particularly in wet environments, and allows a user to visually inspect an endovascular device disposed within the introducer sheath prior to use of the endovascular device. It would also be desirable to provide a tubular introducer sheath that allows a user to visually inspect any features of interest on such an endovascular device within the introducer sheath by axially sliding the introducer sheath so that the features of interest can be seen, without having to rotate the introducer sheath to inspect the endovascular device. The present invention meets these and other needs.

### SUMMARY OF THE INVENTION

Briefly and in general terms, the present invention provides for a tubular introducer sheath made of predominantly clear material, with a colored stripe that extends along the length of the tubular introducer sheath and that in a presently preferred aspect, spirals along the length of the tubular introducer sheath around the longitudinal axis of the tubular introducer sheath.

The present invention accordingly provides for a tubular introducer sheath for introducing an endovascular medical device into a catheter. In a presently preferred embodiment, the tubular introducer sheath includes a hollow, elongated tubular member having an outer side wall with a predominantly substantially clear portion, and a colored stripe embedded in the substantially clear portion of the outer side wall of the hollow, elongated tubular member along the length of the hollow, elongated tubular member. In a presently preferred aspect, the colored stripe spirals around the hollow, elongated tubular member and along the longitudinal axis of the hollow, elongated tubular member. In another presently preferred aspect, the colored stripe has a width that is sufficiently narrow that the clear area allows visual inspection through the tubular introducer sheath. In another presently preferred aspect, the distal end portion of the hollow, elongated tubular member is tapered.

The present invention also provides for a system for introducing an endovascular medical device into a catheter, including a hollow, elongated tubular introducer sheath having an outer side wall with a predominantly substantially clear portion, and a colored stripe embedded in the substantially clear portion of the outer side wall of the hollow, elongated tubular introducer sheath along the length of the hollow, elongated tubular introducer sheath, and an endovascular medical device removably disposed within the hollow, elongated tubular introducer sheath. In a presently preferred aspect, the colored stripe spirals around the hollow, elongated tubular member and along the longitudinal axis of the hollow, elongated tubular introducer sheath. In another presently preferred aspect, the colored stripe has a width that is sufficiently narrow that the clear area allows visual inspection through the hollow, elongated tubular introducer sheath. Inspection of a device within the hollow, elongated tubular introducer sheath through the clear portions of the introducer sheath is easily done by axially sliding the introducer sheath so that features of interest are not blocked by the colored stripe, without the need to rotate the hollow, elongated tubular introducer sheath to inspect the device. In another presently preferred aspect, the distal end portion of the hollow, elongated tubular introducer sheath is tapered.

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

FIG. 1 is a side elevational view of a striped tubular stent introducer sheath, according to the invention.

FIG. 2 is a cross-sectional view taken along line 2-2 of FIG. 1.

FIG. 3 is a side elevational view of a variation of the striped tubular stent introducer sheath of FIG. 1.

FIG. 4 is a cross-sectional view taken along line 4-4 of FIG. 3.

### DETAILED DESCRIPTION OF THE PREFERRED EMBODIMENTS

Referring to the drawings, which are provided by way of example, and not by way of limitation, the present invention provides for a tubular introducer sheath 10 for introducing an endovascular medical device 12 into a catheter (not shown). The tubular introducer sheath is preferably formed as a hollow, elongated tubular member 14 having a proximal end 16, a distal end 18, and a length 20 extending between the proximal and distal ends. The tubular introducer sheath includes an outer side wall 22, a longitudinal axis 24, and a longitudinal interior channel 26 configured to removably contain the endovascular medical device therewithin, for introducing the endovascular medical device into a catheter.

In a presently preferred aspect, the tubular introducer sheath is made of predominantly clear material, such as a clear plastic or polymeric material, such as polytetrafluoroethylene (PTFE) or polycarbonate, for example, forming a clear portion 28 with a colored stripe 30 embedded in the outer side wall of the hollow, elongated tubular member along the length of the hollow, elongated tubular member.

The colored stripe is preferably formed to spiral around along the longitudinal axis of the tubular introducer sheath. During extrusion of the tubular introducer sheath, a discrete sector of the circumference is displaced by a stream of colored material. As the extrusion proceeds, the material is rotated so that the stripe of colored material travels around the tube. The width of the stripe preferably is formed to be sufficiently wide to be visible externally, and sufficiently narrow that the clear portion of the tubular introducer sheath allows visual inspection of an endovascular medical device disposed in the tubular introducer sheath through the clear portion of the tubular introducer sheath. The presence of the colored stripe helps the user determine if the end of the tubular introducer sheath has been inserted all the way to the bottom of the tapered hub opening.

In another presently preferred aspect, in one variation, a distal end portion 32 of the tubular introducer sheath can be tapered, such as to match an internal taper of a catheter hub through which the endovascular medical device may be introduced into a catheter.

As is illustrated in the drawings, when a sample device is within the spiral-striped introducer sheath, the ends of the introducer sheath are clearly marked by the termination of the colored stripe. Inspecting the device through the clear portions of the introducer sheath is easily done by axially sliding the introducer sheath so that the features of interest are not blocked by the stripe, and it is not necessary to rotate the introducer sheath to inspect the device.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A tubular introducer sheath for introducing an endovascular medical device into a catheter, the tubular introducer sheath comprising:
a hollow, elongated tubular member having a proximal end, a distal end, a length extending between the proximal and distal ends, an outer side wall, a longitudinal axis, and a longitudinal interior channel configured to removably contain an endovascular medical device therewithin, said outer side wall having a predominantly substantially clear portion; and
a colored stripe embedded in the substantially clear portion of said outer side wall of the hollow, elongated tubular member along the length of the hollow, elongated tubular member.

2. The tubular introducer sheath of claim 1, wherein said colored stripe spirals around the hollow, elongated tubular member and along the longitudinal axis of the hollow, elongated tubular member.

3. The tubular introducer sheath of claim 1, wherein said colored stripe has a width that is sufficiently narrow that the clear area allows visual inspection through the tubular introducer sheath.

4. The tubular introducer sheath of claim 1, wherein a distal end portion of the hollow, elongated tubular member is tapered.

5. A system for introducing an endovascular medical device into a catheter, comprising:
a hollow, elongated tubular introducer sheath having a proximal end, a distal end, a length extending between the proximal and distal ends, an outer side wall, a longitudinal axis, and a longitudinal interior channel, said outer side wall having a predominantly substantially clear portion;
a colored stripe embedded in the substantially clear portion of said outer side wall of the hollow, elongated tubular introducer sheath along the length of the hollow, elongated tubular introducer sheath; and
an endovascular medical device removably disposed within said hollow, elongated tubular introducer sheath.

6. The system of claim 5, wherein said colored stripe spirals around the hollow, elongated tubular member and along the longitudinal axis of the hollow, elongated tubular introducer sheath.

7. The system of claim 5, wherein said colored stripe has a width that is sufficiently narrow that the clear area allows visual inspection through the hollow, elongated tubular introducer sheath.

8. The system of claim 5, wherein a distal end portion of the hollow, elongated tubular introducer sheath is tapered.
